(19) United States
(12) Patent Application Publication
(10) Pub. No.: US 2012/0010655 A1
(43) Pub. Date: Jan. 12, 2012

(54) SUTURE DEVICE WITH ADJUSTABLE CURVED NEEDLE

(76) Inventor: Hsieh-Hsing LIN, NEW TAIPEI CITY (TW)

(21) Appl. No.: 13/174,812

(22) Filed: Jul. 1, 2011

(30) Foreign Application Priority Data
Jul. 9, 2010 (TW) 099213146

(51) Int. Cl. A61B 17/06

(52) U.S. Cl. 606/223

(57) ABSTRACT

The suture device in accordance with the present invention has a curved needle and a suture thread. The curved needle has a tailing section and a leading section. The tailing section is made from a low-rigid material. The leading section is connected to the tailing section, made from a high-rigid material and has a point portion formed at an end of the leading section opposite to the tailing section. The suture thread is connected to an end of the tailing section opposite to the leading section.

### BACKGROUND OF THE INVENTION

1. Field of the Invention

The present invention relates to a suture device, especially to a surgical suture device with an adjustable curbed needle.

2. Description of the Prior Art

After surgery, for treating a surgery incision made to a tissue or an organ, a conventional suture device is employed to close the incision in order to facilitate recovering of the incision.

With reference to FIG. 6, a conventional suture device 90 comprises a needle 91 and a thread 92. The needle 91 is curved. The thread 92 is connected to an end of the needle 91. The needle 91 of the conventional suture device 90 is made from a high-rigid material to maintain a rigidity of the needle 91 necessary for penetrating a tissue with the needle 91.

However, the needle 91 made from the high-rigid material may not be easily curved and thus it is impossible to adjust the curvature of the needle 91 to fulfill the needs of various surgeries.

To overcome the shortcomings, the present invention provides a suture device to mitigate or obviate the aforementioned problems.

### SUMMARY OF THE INVENTION

The main objective of the invention is to provide a suture device with an adjustable curved needle.

The suture device in accordance with the present invention has a curved needle and a suture thread. The curved needle has a tailing section and a leading section. The tailing section is made from a low-rigid material. The leading section is connected to the tailing section, made from a high-rigid material and has a point portion formed at an end of the leading section opposite to the tailing section. The suture thread is connected to an end of the tailing section opposite to the leading section.

With the foregoing structure of the suture device in accordance with the present invention, specifically the curved needle having the low-rigid tailing section and the high-rigid leading section, the curvature of the curved needle of the suture device may be adjusted according to the needs of various surgeries.

Other objectives, advantages and novel features of the invention will become more apparent from the following detailed description when taken in conjunction with the accompanying drawings.

### BRIEF DESCRIPTION OF THE DRAWINGS

FIG. 1 is a cross sectional side view of a suture device in accordance with the present invention in section;

FIG. 2 is a cross sectional side view of the suture device in FIG. 1 with an adjusted curvature;

FIG. 3 is an operational side view of the suture device in FIG. 1;

FIG. 4 is an operational perspective view of the suture device in FIG. 1 in a process for adjusting the curvature;

FIG. 5 is an operational cross sectional side view of the suture device in FIG. 1 in a process for adjusting the curvature in partial section; and

FIG. 6 is a side view of a conventional suture device in accordance with the prior art.

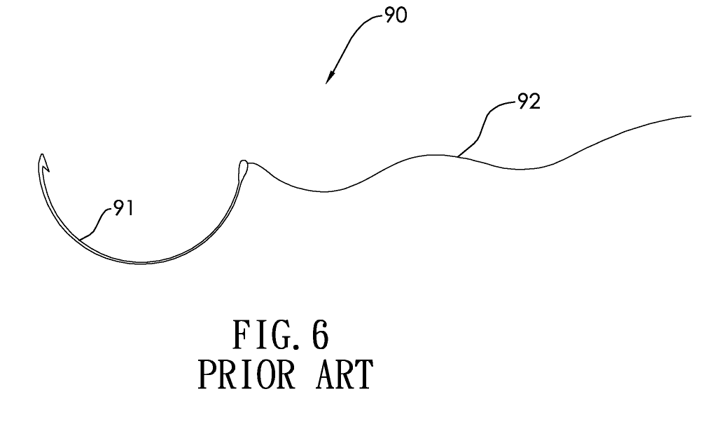

### DETAILED DESCRIPTION OF THE PREFERRED EMBODIMENTS

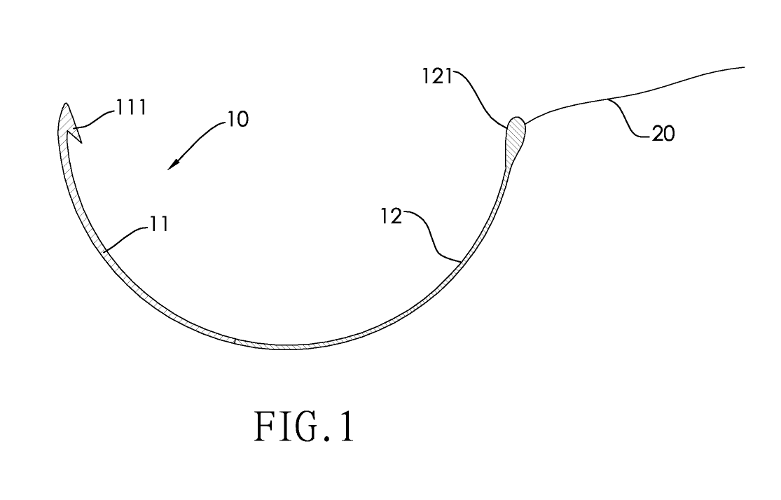

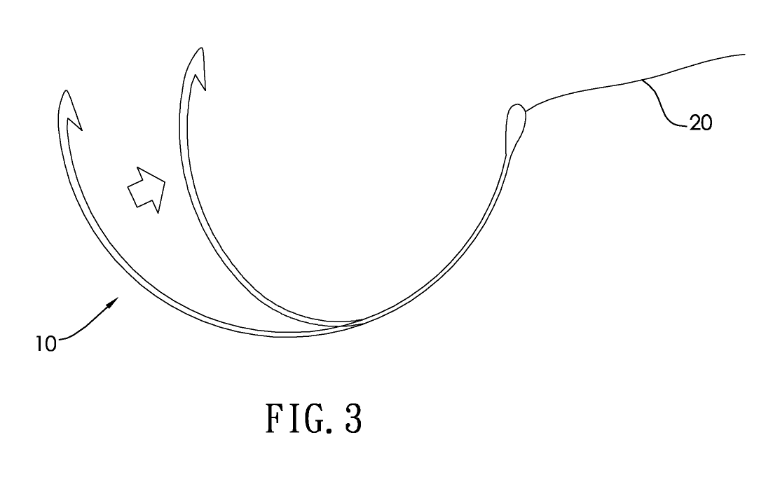

With reference to FIGS. 1, 2 and 3, a suture device with an adjustable curbed needle in accordance with the present invention comprises a curved needle 10 and a suture thread 20.

The curved needle 10 comprises a tailing section 12 and a leading section 11. The leading section 11 is connected to the tailing section 12 and comprises a point portion 111 and is made from a high-rigid material for penetrating a tissue with the needle 91. The tailing section 12 is made from a low-rigid material and is bendable for adjustment of curvature thereof. In the instant embodiment, the curved needle 10 further comprises a connecting portion 121 formed at an end of the tailing section 12 opposite to the leading section 11.

The suture thread 20 has an end connected to the connecting portion 121 of the tailing section 12 of the curved needle 10. In the instant embodiment, the suture thread 20 is tied to the connecting portion 121.

It is to be noted that the although the leading section 11 is made from a high-rigid material so to maintain a rigidity for penetrating a tissue with the curved needle 10, the tailing section 12 of the curved needle 10 is made from a low-rigid material and the curvature of the tailing section 12 may be easily adjusted. One embodiment of the tailing section 12 is made from Stainless steel with TMA and nickel titanium (NiTi). Other materials suitable for rendering the tailing section 12 bendable may also be employed in the manufacture of the tailing section 12. In a process for making the suture curved needle 10, the tailing section 12 may be quenched to acquire the bendable characteristic. The tailing section 12 may be marked with a color or colors so as to provide user-friendliness.

With the foregoing structure, with in a surgery, a surgeon may adjust the curvature of the curved needle 10 according to the needs of the surgery by bending the tailing section 12 of the curved needle 10.

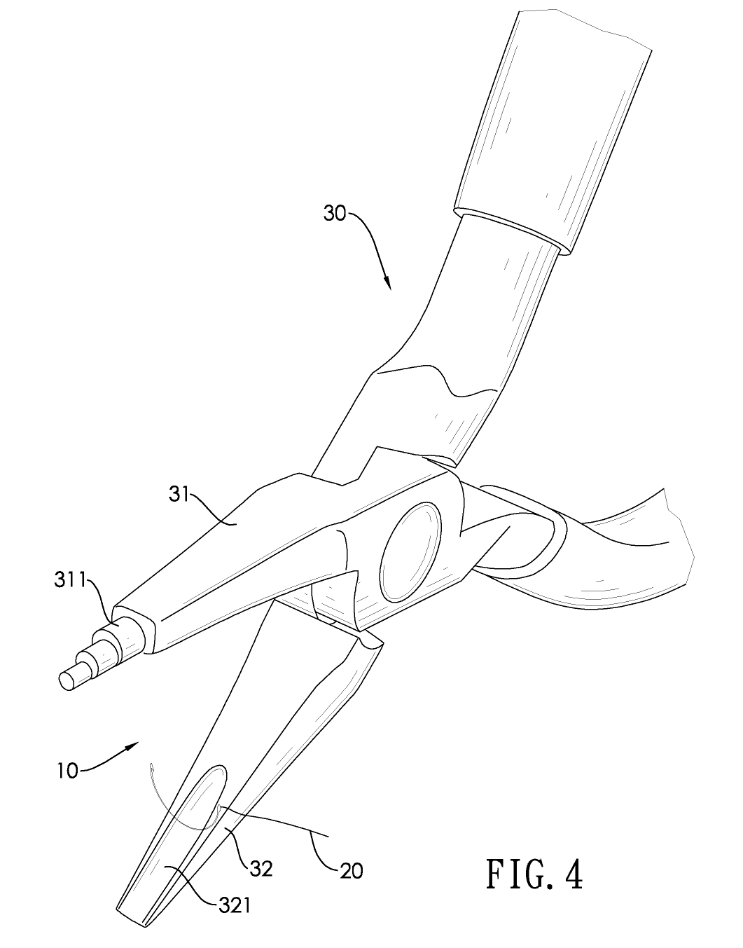

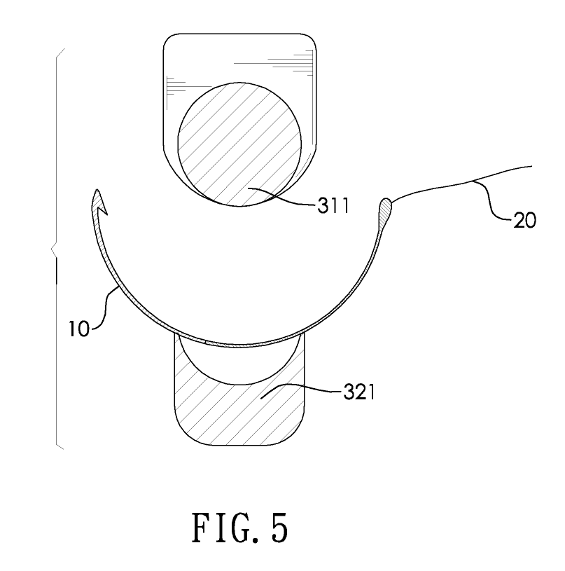

With reference to FIGS. 1, 4 and 5, at least two means may be employed to bend the tailing section 12 of the curved needle 10.

The first means is performed with pliers 30 comprising an upper clamping lever 31 and a lower clamping lever 32. A fore portion of the upper clamping lever 31 comprises multiple cylinders 311 forming a shape of different levels. Each of the cylinders 311 has a smaller diameter than the diameter of a proximal neighboring cylinder.

The lower clamping lever 32 comprises an arced slot 321 corresponding to the cylinders 311 of the upper clamping lever 31.

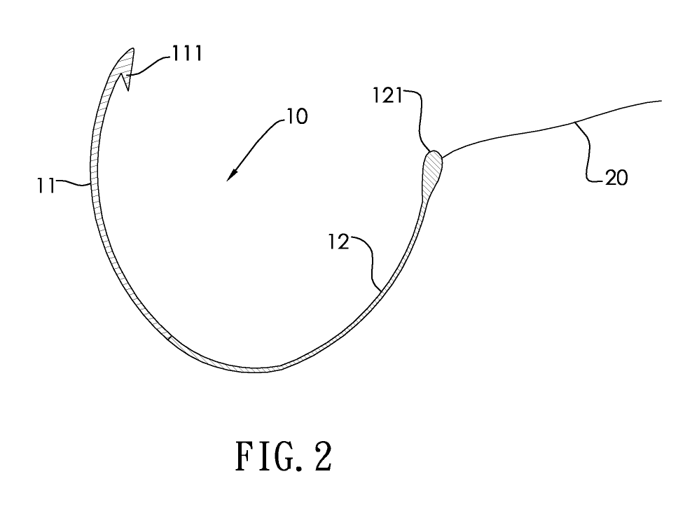

While adjusting the curvature of the curved needle 10, the tailing section 12 of the needle 10 is placed between the cylinders 311 of the upper clamping lever 31 and the arced slot 321 of the lower clamping lever 32 of the clamp 30, facing two endpoints of the needle 10 to the cylinders 311. The clamp 30 is then closed to received the cylinders 311 of the upper clamping lever 31 in the arced slot 321 of the lower clamping lever 32 to bend the tailing section 12 of the curved needle 10, so as to adjust the curvature of the curved needle 10 from an original state as shown in FIG. 1 to an further curved state as shown in FIG. 2, or to a desired curved state of curvature of ⅜, 4/8 or ⅝ circle.

The second means is performed with a temperature change. A curved needle 10 suitable for practicing the second means is one that has been heat-processed so as to equip the tailing section 12 of the curved needle 10 with a special material characteristic. For example, a temperature change may be applied to the curved needle 10 by immersing the curved needle 10 into icy water. When being immersed in the icy water, a material characteristic of the tailing section 12 of the curved needle 10 causes a one-time change to alter the curvature of the curved needle 10 from an original state as shown in FIG. 1 to an further curved state as shown in FIG. 2, such that when returned to room temperature, that material characteristic of the tailing section 12 of the curved needle 10 prevent the curvature of the curved needle 10 from returning to the original state.

With the foregoing structure of the suture device in accordance with the present invention, specifically the curved needle 10 having the low-rigid tailing section 12 and the high-rigid leading section 11, the curvature of the curved needle 10 of the suture device may be adjusted according to the needs of various surgeries.

What is claimed is:

1. A suture device comprising
a curved needle comprising
a tailing section made from a low-rigid material and being bendable and
a leading section connected to the tailing section, made from a high-rigid material and comprising
a point portion formed at an end of the leading section opposite to the tailing section, and
a suture thread connected to an end of the tailing section opposite to the leading section.

2. The suture device as claimed in claim 1, wherein the curved needle further comprises a connecting portion formed at the end of the tailing section opposite to the leading section, and
the suture thread is connected to the connecting portion of the tailing section of the curved needle.
